(19) United States
(12) Patent Application Publication
(10) Pub. No.: US 2025/0176988 A1
(43) Pub. Date: Jun. 5, 2025

(54) FORCEPS AND FORCEPS SYSTEM

(71) Applicant: CODEN CO., LTD., Tokyo (JP)

(72) Inventors: Kojiro Koda, Tokyo (JP); Masaaki Tomimizu, Tokyo (JP); Nobumi Tagaya, Kitakatsushika-gun (JP)

(73) Assignee: CODEN CO., LTD., Tokyo (JP)

(21) Appl. No.: 18/920,421

(22) Filed: Oct. 18, 2024

(30) Foreign Application Priority Data
Dec. 5, 2023 (JP) 2023-205245

(51) Int. Cl. A61B 17/29

(52) U.S. Cl. CPC A61B 17/29

(57) ABSTRACT

This disclosure provides a forceps that is easily used integrally with an endoscope. A forceps 1 includes: a forceps insertion unit 21; and a treatment unit 41 configured to perform a predetermined treatment, the treatment unit 41 being disposed in the forceps insertion unit 21 and protruding toward a distal end side with respect to the forceps insertion unit 21, in which the forceps insertion unit 21 includes a large-diameter cylindrical portion 22 in which a proximal end side portion of the treatment unit 41 is disposed, and a small-diameter cylindrical portion 24 which is disposed in the large-diameter cylindrical portion 22 and into which an endoscope insertion unit 11 for observing an outside of an endoscope 10 is insertable.

### FIELD OF THE INVENTION

The present invention relates to a forceps and a forceps system.

### BACKGROUND OF THE INVENTION

In the related art, in the medical field, forceps having various shapes and sizes are used for various purposes (refer to, for example, Japanese Patent Publication No. 2022-181850). However, it is difficult to use the forceps with the endoscope integrally.

In recent years, low-invasive endoscopic surgery or laparoscopic surgery has become mainstream to reduce the burden on the patient's body. In particular, in a surgical operation of a digestive organ, the number of laparotomies in the related art have been significantly reduced, and laparoscopic surgery account for approximately 70%. In laparoscopic surgery, a plurality of holes through which a camera (main endoscope) or a forceps is passed are made in the abdominal part of the patient.

Laparoscopic surgery has a lot of advantages compared to the laparotomy in the related art. These advantages include that the number of complications in a patient after surgery is small, the suturing range is small, and the number of hospitalization days can be significantly reduced.

In addition, a doctor who performs the laparoscopic surgery does not need to take a forward-bending posture for a long time to look into the abdominal cavity during the surgery. Further, since the camera enlarges and reflects the affected part on the monitor, the doctor can make a detailed determination of the state of the affected part and does not need to make a painful forward-bending posture. However, in laparoscopic surgery, a camera has blind spots. Thus, there is a need to develop methods for reducing these blind spots.

### SUMMARY OF THE INVENTION

The present disclosure has been made in view of the above-described issue, and an object of the present disclosure is to provide a forceps that is easily used integrally with an endoscope, and a forceps system including the forceps.

In one aspect, provided is a forceps comprising:
a forceps insertion unit; and
a treatment unit configured to perform a predetermined treatment, the treatment unit being disposed in the forceps insertion unit and protruding toward a distal end side with respect to the forceps insertion unit, wherein
the forceps insertion unit includes
a large-diameter cylindrical portion in which a proximal end side portion of the treatment unit is disposed, and
a small-diameter cylindrical portion which is disposed in the large-diameter cylindrical portion and into which an endoscope insertion unit for observing an outside of an endoscope is insertable,
the treatment unit includes two gripping pieces that are openable and closable relative to each other,
distal end portions of the two gripping pieces are opened and closed on a reference plane along an axis of the large-diameter cylindrical portion, and
an entire opening at a distal end of the small-diameter cylindrical portion is disposed on any side perpendicular to the reference plane with respect to the reference plane.

In one embodiment, for example, in a laparoscopic surgery, the endoscope insertion unit of the endoscope is inserted into the small-diameter cylindrical portion of the forceps. In this state, a distal end side portion of the large-diameter cylindrical portion is inserted into the abdominal part of the patient while observing the outside of the endoscope insertion unit such as a distal end side portion with respect to the large-diameter cylindrical portion by the endoscope insertion unit. Then, the treatment unit performs the predetermined treatment.

Since the endoscope insertion unit can be inserted into the small-diameter cylindrical portion, the endoscope insertion unit can be easily inserted into the forceps insertion unit, and the forceps can be easily used integrally with the endoscope.

In addition, the endoscope insertion unit observes the distal end side with respect to the large-diameter cylindrical portion through the opening of the small-diameter cylindrical portion. In a case where the predetermined treatment is performed by opening and closing the two gripping pieces, the entire opening is disposed on any side perpendicular to the reference plane with respect to the reference plane on which the distal end portions of the two gripping pieces are opened and closed. Therefore, when the observation is performed by the endoscope insertion unit through the opening, it is possible to prevent the distal end portions of the two gripping pieces from interfering with the observation.

In one embodiment, the forceps described herein further comprises an opening and closing regulation unit configured to regulate an opening and closing range of the two gripping pieces.

In one embodiment, the opening and closing regulation unit can suppress the damage of the two gripping pieces due to the excessive force applied to the two gripping pieces which are opened and closed.

In one embodiment, the forceps described herein includes a fixing unit that is disposed at an intermediate portion of the large-diameter cylindrical portion in an axis direction of the large-diameter cylindrical portion and fixes a position of the small-diameter cylindrical portion with respect to the large-diameter cylindrical portion.

In one embodiment, the fixing unit can hold the position of the small-diameter cylindrical portion with respect to the large-diameter cylindrical portion in a portion where the intensity is relatively decreased, that is, the intermediate portion in the axis direction.

In one embodiment, the treatment unit extends along a treatment axis along the large-diameter cylindrical portion, and the forceps further includes a rotating operation unit configured to rotate the treatment unit around the treatment axis.

In one embodiment, by operating the rotating operation unit, a direction around the treatment axis in which a predetermined treatment is performed by the treatment unit can be changed, for example, without changing a direction of an external field of view by the endoscope insertion unit. In one embodiment, the forceps described herein further comprises an attachment unit to which an endoscope main body of the endoscope is attached. In one embodiment, the attachment unit attaches the endoscope main body of the endoscope to the forceps, so that the operator such as a doctor can operate the forceps and the endoscope main body integrally.

In one embodiment, the forceps described herein further comprises a rotation regulation unit configured to regulate a direction of the endoscope insertion unit around a small-diameter axis of the small-diameter cylindrical portion. In one embodiment the rotation regulation unit can suppress change in a direction of an external field of view by the endoscope insertion unit due to the rotation of the endoscope insertion unit around the small-diameter axis.

In one embodiment, the forceps described herein further comprises an insertion regulation unit configured to regulate a position of the endoscope insertion unit disposed in the small-diameter cylindrical portion in a small-diameter axis direction of the small-diameter cylindrical portion. In one embodiment, the insertion regulation unit can suppress the change in the position of the endoscope insertion unit in the small-diameter axis direction with respect to the small-diameter cylindrical portion.

In one embodiment, provided is a forceps system comprising the forceps described herein and the endoscope. In one embodiment, the forceps system can be configured by using the forceps that is easily used integrally with the endoscope. In one embodiment, the forceps and the forceps system described herein can be easily used integrally with the endoscope.

### BRIEF DESCRIPTION OF THE FIGURES

FIG. 1 is a partially fragmented perspective view of a forceps system according to an embodiment of the present invention.

FIG. 2 is a cross-sectional view taken along line A1-A1 of FIG. 1.

FIG. 3 is a view taken in a direction of an arrow A2 of FIG. 1.

FIG. 4 is a plan view of a main part of forceps in the forceps system.

FIG. 5 is a view explaining a procedure using the forceps system.

FIG. 6 is a plan view of a main part in a forceps system of a first modification example according to the embodiment of the present invention.

FIG. 7 is a cross-sectional view of a main part in a forceps system of a third modification example according to the embodiment of the present invention.

### DETAILED DESCRIPTION OF THE INVENTION

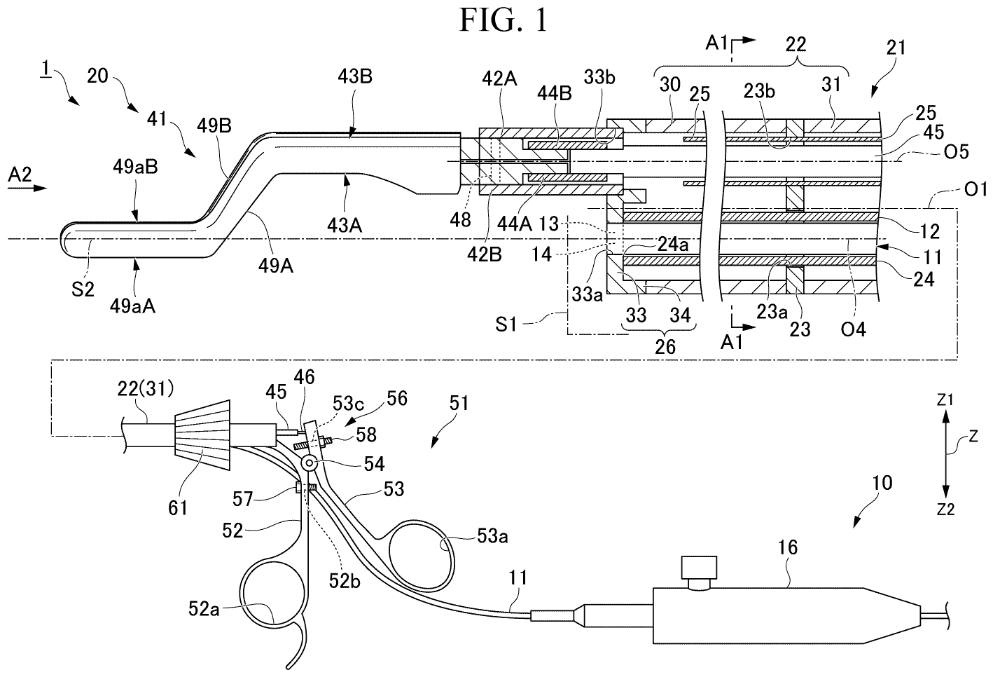

Provided herein is an embodiment of a forceps and a forceps system according to the present invention that is described with reference to FIGS. 1 to 7.

As shown in FIG. 1, a forceps system 1 according to the present embodiment includes a sub-endoscope (endoscope) 10 and a forceps 20 according to the present embodiment. The sub-endoscope 10 includes an endoscope insertion unit 11, an endoscope main body 16, and a display unit (not shown).

Hereinafter, a side of the endoscope insertion unit 11 with respect to the endoscope main body 16 (a side of the forceps insertion unit 21 with respect to an opening and closing operation unit 51 described below in the forceps 20) is referred to as a distal end side. A side of the endoscope main body 16 with respect to the endoscope insertion unit 11 (a side of the opening and closing operation unit 51 with respect to the forceps insertion unit 21 described below in the forceps 20) is referred to as a proximal end side.

For example, the endoscope insertion unit 11 includes an outer sheath tube 12, a light projecting unit 13, and an imaging unit 14.

The outer sheath tube 12 is formed in a tubular shape and has flexibility. The light projecting unit 13 and the imaging unit 14 are fixed inside the distal end portions of the outer sheath tube 12.

The light projecting unit 13 includes a light emitting diode (LED) (not shown) or the like. The light projecting unit 13 emits visible light toward the distal end side. A power supply line (not shown) is connected to the light projecting unit 13. The power supply line extends toward the proximal end side through the outer sheath tube 12. A predetermined power is supplied to the light projecting unit 13 through the power supply line.

The imaging unit 14 includes a light-receiving element, such as a complementary metal-oxide-semiconductor (CMOS) (not shown). The imaging unit 14 converts visible light reflected to an object to be observed, such as a tissue of a patient, into an image. The imaging unit 14 converts the image into a signal. A signal line (not shown) is connected to the imaging unit 14. The signal line extends toward the proximal end side through the outer sheath tube 12. The imaging unit 14 sends the signal through the signal line.

As described above, the endoscope insertion unit 11 is a device for observing the outside of the endoscope insertion unit 11, such as the distal end side of the endoscope insertion unit 11.

The endoscope main body 16 is connected to a proximal end portion of the outer sheath tube 12. The power supply line and the signal line are each connected to the endoscope main body 16. For example, the endoscope main body 16 supplies the predetermined power to the power supply line and receives the signal sent by the imaging unit 14 from the signal line. The endoscope main body 16 appropriately processes the received signal for the display unit.

The display unit includes a liquid crystal panel and is connected to the endoscope main body 16. The display unit displays the image based on the appropriately processed signal.

Next, the forceps 20 will be described.

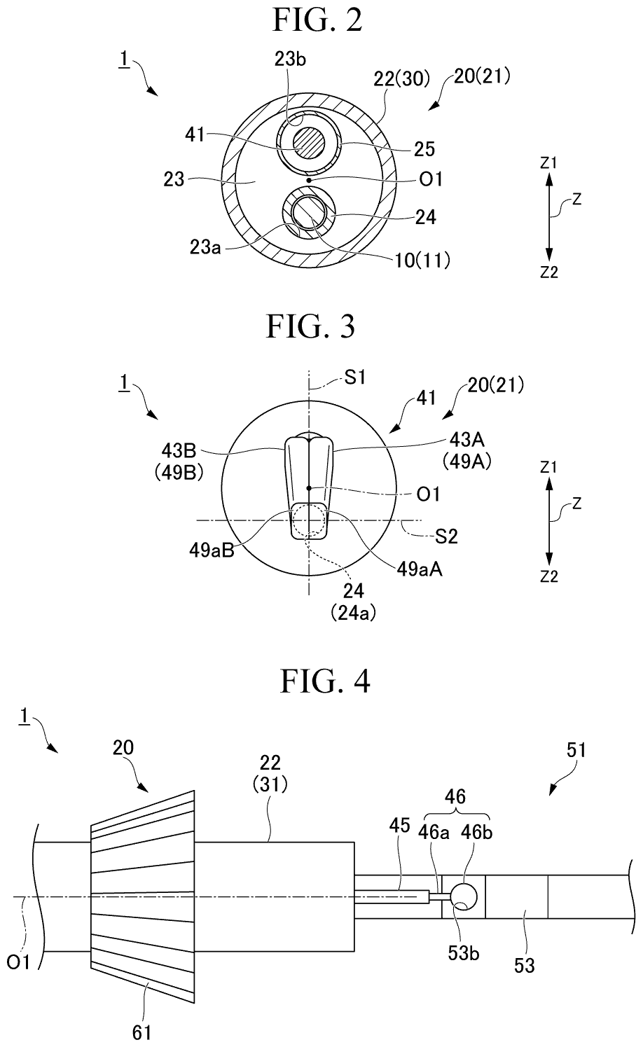

The forceps 20 includes the forceps insertion unit 21, a treatment unit 41, the opening and closing operation unit 51, an opening and closing regulation unit 56, and a rotating operation unit 61. As shown in FIGS. 1 and 2, the forceps insertion unit 21 includes a large-diameter cylindrical portion 22, a fixing unit 23, a first small-diameter cylindrical portion (small-diameter cylindrical portion) 24, a second small-diameter cylindrical portion 25, and a bracket 26.

As shown in FIG. 1, the large-diameter cylindrical portion 22 includes a distal end cylindrical piece 30 and a proximal end cylindrical piece 31. An inner diameter of the distal end cylindrical piece 30 and an inner diameter of the proximal end cylindrical piece 31 are equal to each other. An outer diameter of the distal end cylindrical piece 30 and an outer diameter of the proximal end cylindrical piece 31 are equal to each other. A length of the distal end cylindrical piece 30 and a length of the proximal end cylindrical piece 31 are equal to each other. The distal end cylindrical piece 30 and the proximal end cylindrical piece 31 are coaxially disposed.

The distal end cylindrical piece 30 is disposed on a distal end side with respect to the proximal end cylindrical piece 31.

The length of the distal end cylindrical piece 30 and the length of the proximal end cylindrical piece 31 may be different from each other.

Hereinafter, the common axis (axis of the large-diameter cylindrical portion 22) of the distal end cylindrical piece 30 and the proximal end cylindrical piece 31 is referred to as an axis O1. A direction perpendicular to the axis O1 is referred to as a radial direction, and a direction rotating around the axis O1 is referred to as a circumferential direction.

A through-hole (not shown) penetrating the proximal end cylindrical piece 31 in the radial direction is formed in the proximal end cylindrical piece 31. The through-hole extends over a predetermined range in the circumferential direction.

As shown in FIGS. 1 and 2, the fixing unit 23 is formed in a disk shape having an outer diameter equal to the outer diameter of the distal end cylindrical piece 30. The fixing unit 23 is disposed on the axis O1 between the distal end cylindrical piece 30 and the proximal end cylindrical piece 31. Through-holes 23a and 23b are formed in the fixing unit 23. The through-holes 23a and 23b penetrate the fixing unit 23 in an axis O1 direction. For example, the through-holes 23a and 23b are disposed across the axis O1 in the radial direction.

The fixing unit 23 is fixed to the distal end cylindrical piece 30 and the proximal end cylindrical piece 31 by welding. It should be noted that a method of fixing the fixing unit 23 and the distal end cylindrical piece 30 and the proximal end cylindrical piece 31 is not limited to welding, and adhesion, compression, or the like may be used.

The fixing unit 23 is disposed at the intermediate portion of the large-diameter cylindrical portion 22 in the axis O1 direction.

The first small-diameter cylindrical portion 24 and the second small-diameter cylindrical portion 25 are each formed in a tubular shape. An outer diameter of the first small-diameter cylindrical portion 24 and an outer diameter of the second small-diameter cylindrical portion 25 are smaller than the inner diameter of the distal end cylindrical piece 30 and the inner diameter of the proximal end cylindrical piece 31. The first small-diameter cylindrical portion 24 and the second small-diameter cylindrical portion 25 are disposed in the distal end cylindrical piece 30 and the proximal end cylindrical piece 31 of the large-diameter cylindrical portion 22.

The first small-diameter cylindrical portion 24 is disposed in the through-hole 23a of the fixing unit 23. The second small-diameter cylindrical portion 25 is disposed in the through-hole 23b of the fixing unit 23. The fixing unit 23 fixes positions of the first small-diameter cylindrical portion 24 and the second small-diameter cylindrical portion 25 with respect to the large-diameter cylindrical portion 22.

As shown in FIG. 1, the bracket 26 includes a flat plate portion 33 and a cylindrical portion 34.

The flat plate portion 33 is formed in a disk shape and is disposed on the axis O1. An outer diameter of the flat plate portion 33 is equal to an outer diameter of the distal end cylindrical piece 30. Through-holes 33a and 33b are formed in a flat plate portion 33 coaxially with the through-holes 23a and 23b. An inner diameter of the through-hole 33a is smaller than an inner diameter of the through-hole 23a.

The cylindrical portion 34 is formed in a tubular shape. The cylindrical portion 34 protrudes from an outer peripheral edge of the flat plate portion 33 toward the proximal end side. The cylindrical portion 34 is disposed on the distal end side with respect to the distal end cylindrical piece 30 of the large-diameter cylindrical portion 22. A stepped portion (reference numeral is omitted) that is reduced in diameter toward the proximal end side is formed between a peripheral edge portion of the through-hole 33b in the flat plate portion 33 and the cylindrical portion 34.

The cylindrical portion 34 abuts against the distal end cylindrical piece 30 in the axis O1 direction, and is fixed to the distal end cylindrical piece 30 by welding or the like.

The distal end of the first small-diameter cylindrical portion 24 is in contact with the flat plate portion 33 of the bracket 26 from the proximal end side of the flat plate portion 33. A space in the first small-diameter cylindrical portion 24 communicates with the through-hole 33a of the flat plate portion 33. A space in the second small-diameter cylindrical portion 25 communicates with the through-hole 33b of the flat plate portion 33. On the other hand, the distal end of the second small-diameter cylindrical portion 25 is spaced apart from the flat plate portion 33 toward the proximal end side.

The distal end of the first small-diameter cylindrical portion 24 is fixed to the flat plate portion 33 by welding or the like.

Here, a first axis (small-diameter axis) O4, which is an axis of the first small-diameter cylindrical portion 24, and a second axis O5, which is an axis of the second small-diameter cylindrical portion 25, are defined.

A first reference plane S1 including the first axis O4 and the second axis O5 is defined. A direction, which is perpendicular to the axes O4 and O5, across the first reference plane S1 is defined as a perpendicular direction Z.

In the perpendicular direction Z, a side of the second axis O5 with respect to the first axis O4 is defined as a first side Z1. A side of the first axis O4 with respect to the second axis O5 is defined as a second side Z2.

A second reference plane (reference plane) S2, which is perpendicular to the first reference plane S1, along the axis O1 is defined. The second reference plane S2 may be a plane intersecting the first reference plane S1 along the axis O1. The forceps system 1 is used such that the first reference plane S1 extends along an up-down direction. In this case, the first side Z1 is disposed upward.

The endoscope insertion unit 11 of the sub-endoscope 10 can be inserted into the first small-diameter cylindrical portion 24.

The forceps 20 may include a proximal end bracket that fixes the positions of the large-diameter cylindrical portion 22, the first small-diameter cylindrical portion 24, and the second small-diameter cylindrical portion 25 on the proximal end side of the forceps insertion unit 21.

As shown in FIGS. 1 and 3, the treatment unit 41 has a well-known link structure. The treatment unit 41 includes support members 42A and 42B, first swing members 43A and 43B, second swing members 44A and 44B, an operation member 45, and a coupling member 46 (refer to FIG. 4).

As shown in FIG. 1, the support members 42A and 42B are formed in a rod shape and extend in the axis O1 direction. The support member 42A is disposed on the first side Z1 with respect to the support member 42B. A gap is formed between the support member 42A and the support member 42B.

A proximal end side portion of the support members 42A and 42B is fixed to the stepped portion of the bracket 26.

As shown in FIGS. 1 and 3, the first swing members 43A and 43B each extend as a whole in the axis O1 direction. The first swing members 43A and 43B are rotationally movable via a first shaft member 48 provided at proximal end portions of each of the first swing members 43A and 43B. The first shaft member 48 extends in the perpendicular direction Z. Both end portions of the first shaft member 48 in the perpendicular direction Z are supported by distal end portions of the support members 42A and 42B.

A distal end side portion of the first swing member 43A with respect to the first shaft member 48 constitutes the gripping piece 49A. A distal end side portion of the first swing member 43B with respect to the first shaft member 48 constitutes the gripping piece 49B. That is, the treatment unit 41 includes two gripping pieces 49A and 49B.

The gripping pieces 49A and 49B can be opened spaced apart from each other, and can be closed in contact with each other. That is, the gripping pieces 49A and 49B can be opened and closed to each other.

Here, for example, from each of distal ends in the gripping pieces 49A and 49B, a range of (¼) of the length of the gripping pieces 49A and 49B in the axis O1 direction is defined as distal end portions 49aA and 49aB of the gripping pieces 49A and 49B. A part of the gripping pieces 49A and 49B is formed in a bent shape. Therefore, in the gripping pieces 49A and 49B, the distal end portions 49aA and 49aB are disposed in a shape that is moved to the distal end side and the second side Z2 with respect to the proximal end portion.

The distal end portions 49aA and 49aB are opened and closed on the second reference plane S2. The opening and closing of the distal end portions 49aA and 49aB on the second reference plane S2 described here means that the distal end portions 49aA and 49aB are opened and closed along the second reference plane S2 in a state where any of the distal end portions 49aA and 49aB is placed on the second reference plane S2.

As shown in FIG. 1, a distal end portion of the second swing member 44A is rotationally movable with respect to a proximal end side portion in the first swing member 43A with respect to the first shaft member 48 via a second shaft member (not shown). A distal end portion of the second swing member 44B is rotationally movable with respect to a proximal end side portion in the first swing member 43B with respect to the first shaft member 48 via a third shaft member (not shown).

The operation member 45 is formed in a rod shape and extends in the axis O1 direction. A proximal end side portion of the operation member 45 is disposed in the second small-diameter cylindrical portion 25. The distal end portion of the operation member 45 is rotationally movable with respect to a proximal end side portion in the second swing member 44A and a proximal end side portion in the second swing member 44B via a fourth shaft member (not shown).

As described above, in the treatment unit 41, the proximal end side portions of the support members 42A and 42B and the operation member 45 are disposed in the second small-diameter cylindrical portion 25 of the forceps insertion unit 21 (large-diameter cylindrical portion 22). The treatment unit 41 extends to the large-diameter cylindrical portion 22 along the second axis O5. In this example, the second axis O5 matches the treatment axis, which is the axis of the treatment unit 41.

The gripping pieces 49A and 49B protrude toward the distal end side of the forceps insertion unit 21 and perform a predetermined treatment such as gripping the tissue of the patient.

In the present embodiment, the opening 24a in the distal end of the first small-diameter cylindrical portion 24 is disposed on the second reference plane S2.

As shown in FIG. 4, the coupling member 46 includes a small-diameter portion 46a and a large-diameter portion 46b. The small-diameter portion 46a is formed in a rod shape and protrudes from the proximal end of the operation member 45 toward the proximal end side. A diameter of the small-diameter portion 46a is smaller than a diameter of the operation member 45.

The large-diameter portion 46b is formed in a spherical shape and is provided at the proximal end of the small-diameter portion 46a. A diameter of the large-diameter portion 46b is larger than a diameter of the small-diameter portion 46a.

As shown in FIG. 1, the opening and closing operation unit 51 includes an operation unit main body 52 and an opening and closing lever 53.

The operation unit main body 52 is fixed to a proximal end portion of the proximal end cylindrical piece 31 of the large-diameter cylindrical portion 22. The operation unit main body 52 extends from the proximal end portion of the proximal end cylindrical piece 31 toward the second side Z2. The distal end portion to which the operation unit main body 52 extends is provided with a first finger hole 52a.

The opening and closing lever 53 is disposed on the proximal end side with respect to the operation unit main body 52 and extends toward a direction between the proximal end side and the second side Z2.

An intermediate portion of the opening and closing lever 53 in a longitudinal direction is connected to the operation unit main body 52 to be rotationally movable by an opening and closing shaft member 54. The opening and closing shaft member 54 is provided at an intermediate portion of the operation unit main body 52 in the perpendicular direction Z. The opening and closing lever 53 rotationally moves on the first reference plane S1 with respect to the operation unit main body 52.

A female screw 52b is formed in a portion on the second side Z2 with respect to a portion, where the opening and closing shaft member 54 is provided, in the operation unit main body 52.

A second finger hole 53a is formed in a portion of the opening and closing lever 53 on the second side Z2. As shown in FIG. 4, a dovetail groove 53b is formed on a surface of the opening and closing lever 53 facing the distal end side in a portion on the first side Z1. The dovetail groove 53b extends along the portion of the opening and closing lever 53 on the first side Z1. The large-diameter portion 46b of the coupling member 46 is fitted into the dovetail groove 53b of the opening and closing lever 53.

As shown in FIG. 1, a female screw 53c is formed in a portion on the first side Z1 with respect to a portion, where the opening and closing shaft member 54 is provided, in the opening and closing lever 53.

The opening and closing regulation unit 56 includes a first regulation unit 57 and a second regulation unit 58.

In this example, screws are used for the first regulation unit 57 and the second regulation unit 58. The first regulation unit 57 is fitted with the female screw 52b. The first regulation unit 57 protrudes from the operation unit main body 52 toward the opening and closing lever 53 side (proximal end side). The second regulation unit 58 is fitted with the female screw 53c. The second regulation unit 58 protrudes from the opening and closing lever 53 toward the operation unit main body 52 side (distal end side).

The protruding amount of the regulation units 57 and 58 can be adjusted by adjusting the position in which the screws are fitted. It should be noted that the opening and closing regulation unit 56 may have only one of the first regulation unit 57 and the second regulation unit 58.

The rotating operation unit 61 is formed in a cylindrical shape and is fitted on the proximal end cylindrical piece 31 of the forceps insertion unit 21. The rotating operation unit 61 is connected to the support members 42A and 42B of the treatment unit 41 via a coupling member (not shown) disposed in the through-hole of the proximal end cylindrical piece 31.

In a case where the rotating operation unit 61 is rotated with respect to the forceps insertion unit 21 around the axis O1, the treatment unit 41 is rotated around the second axis O5. In a case where the rotating operation unit 61 is rotated, the coupling member is locked to the peripheral edge portion of the through-hole in the proximal end cylindrical piece 31 in the circumferential direction, so that the rotation range of the rotating operation unit 61 around the axis O1 is regulated.

Each configuration of the forceps 20 is formed of stainless steel or the like.

For example, the operator grips the opening and closing operation unit 51 of the forceps 20 by hand. At this time, the middle finger is inserted into the first finger hole 52a of the operation unit main body 52, and the thumb is inserted into the second finger hole 53a of the opening and closing lever 53. In a case where the operator grips the opening and closing operation unit 51, the finger holes 52a and 53a approach each other, and the first regulation unit 57 comes into contact with the opening and closing lever 53 from the distal end side of the opening and closing lever 53. The operation member 45 is moved to the proximal end side with respect to the forceps insertion unit 21, and the distal end portions 49aA and 49aB of the gripping pieces 49A and 49B come into contact with each other, whereby the two gripping pieces 49A and 49B are closed. At this time, the large-diameter portion 46b of the coupling member 46 moves along the dovetail groove 53b in the dovetail groove 53b of the opening and closing lever 53.

On the other hand, in a case where the operator opens the opening and closing operation unit 51, the finger holes 52a and 53a are spaced apart from each other, and the second regulation unit 58 comes into contact with the operation unit main body 52 from the proximal end side of the operation unit main body 52. The operation member 45 is moved to the distal end side with respect to the forceps insertion unit 21, and the distal end portions 49aA and 49aB of the gripping pieces 49A and 49B are spaced apart from each other, whereby the two gripping pieces 49A, 49B are opened.

As described above, the first regulation unit 57 and the second regulation unit 58 provided in the opening and closing regulation unit 56 regulate the opening and closing ranges of the gripping pieces 49A and 49B (the ranges from the angles formed by the gripping pieces 49A and 49B when the gripping pieces 49A and 49B are closed to the angles formed by the gripping pieces 49A and 49B when the gripping pieces 49A and 49B are opened).

For example, an outer diameter of the large-diameter cylindrical portion 22 is 5 mm, and a length of the large-diameter cylindrical portion 22 is approximately 30 cm to 40 cm. In the forceps insertion unit 21, the first small-diameter cylindrical portion 24 and the second small-diameter cylindrical portion 25 are disposed in the large-diameter cylindrical portion 22.

Next, an operation of the forceps system 1 configured as described above will be described.

For example, in laparoscopic surgery, the endoscope insertion unit 11 of the sub-endoscope 10 is inserted into the first small-diameter cylindrical portion 24 of the forceps 20. It is preferable that, in a case where the endoscope insertion unit 11 is inserted into the first small-diameter cylindrical portion 24, the distal end of the endoscope insertion unit 11 matches the distal end of the forceps insertion unit 21. It should be noted that, in a case where the endoscope insertion unit 11 is inserted into the first small-diameter cylindrical portion 24, the distal end of the endoscope insertion unit 11 may be disposed on the proximal end side with respect to the distal end of the forceps insertion unit 21.

The endoscope insertion unit 11 of the sub-endoscope 10 can be freely attached to and detached from the forceps 20.

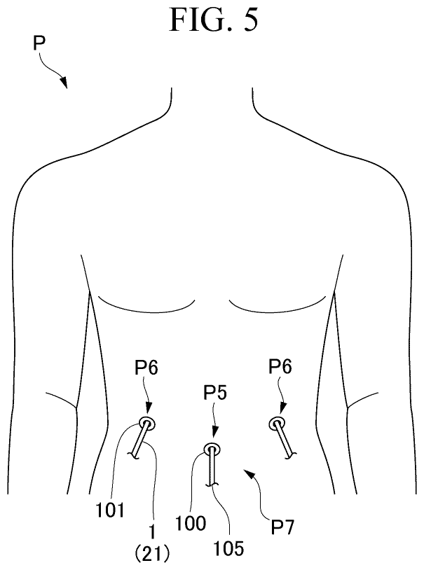

As shown in FIG. 5, for example, holes (not shown) are formed in the umbilical cord (navel) P5 of the patient P and a portion P6 beside the umbilical cord P5. Each of trocars 100 and 101 are attached to each of the holes. The main endoscope 105 is inserted into the abdominal part P7 through the trocar 100. The forceps insertion unit 21 of the forceps system 1 is inserted into the abdominal part P7 through the trocar 101. An image acquired by the main endoscope 105 is appropriately displayed. An image acquired by the sub-endoscope 10 is displayed on the display unit. Carbon dioxide gas is injected into the abdominal part P7 via the trocars 100 and 101 to inflate the abdominal part P7.

In this state, the operator inserts the distal end side portion of the large-diameter cylindrical portion 22 into the abdominal part P7 of the patient P through the trocar 101 while observing the distal end side of the large-diameter cylindrical portion 22 by the endoscope insertion unit 11. Then, the treatment unit 41 performs a predetermined treatment. A part that is difficult to observe with the main endoscope 105, blind spots of the main endoscope 105, and the like can be observed with the sub-endoscope 10.

Since the endoscope insertion unit 11 can be inserted into the first small-diameter cylindrical portion 24, the endoscope insertion unit 11 can be easily inserted into the forceps insertion unit 21, and the forceps 20 can be easily used together with the sub-endoscope 10.

The sub-endoscope 10 and the forceps 20 can be separately managed from each other, so that, for example, storing, washing, and sterilization can be easily performed only by the forceps 20.

The forceps 20 is also a consumable item because it is a non-passive medical device. Therefore, in a case where the movement of the forceps 20 is deteriorated, it is necessary to replace the forceps 20. On the other hand, the sub-endoscope 10 has no movable portion, and thus has durability and does not need to be replaced.

The forceps 20 includes the opening and closing regulation unit 56. Therefore, it is possible to suppress the damage of the gripping pieces 49A and 49B due to the excessive force applied to the gripping pieces 49A and 49B which are opened and closed.

The forceps 20 includes the fixing unit 23. The fixing unit 23 can hold the position of the first small-diameter cylindrical portion 24 with respect to the large-diameter cylindrical portion 22 in a portion where the intensity is relatively decreased, that is, the intermediate portion in the axis O1 direction.

The forceps 20 includes the rotating operation unit 61. Therefore, by operating the rotating operation unit 61, the direction around the second axis O5 in which the predetermined treatment is performed by the treatment unit 41 can be changed, for example, without changing the direction around the first axis O4 of the field of view on the distal end side by the endoscope insertion unit 11.

In addition, in the forceps system 1 according to the present embodiment, the forceps system can be configured by using the forceps 20 that is easily used integrally with the sub-endoscope 10.

The forceps system of the present embodiment can be configured in variously modified manners as described below.

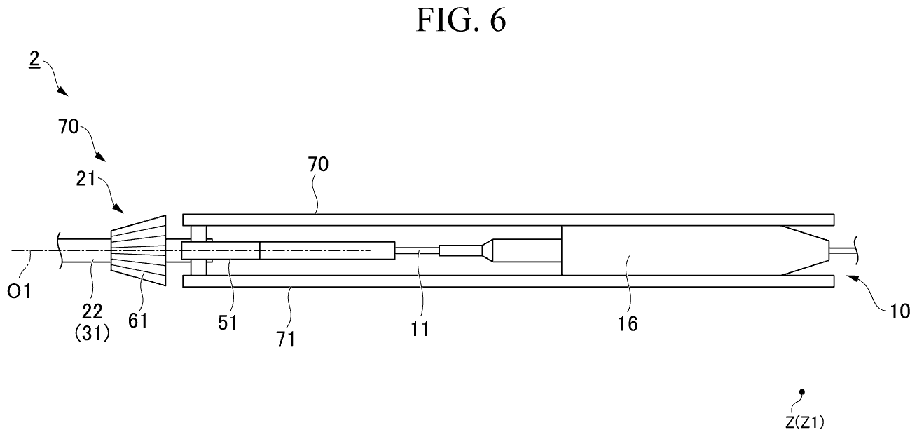

In the forceps system 2 of the first modification example shown in FIG. 6, a forceps 70 is provided instead of the forceps 20 in each configuration of the forceps system 1 according to the present embodiment. The forceps 70 includes an attachment unit 71 in addition to each configuration of the forceps 20.

For example, the attachment unit 71 extends in the axis O1 direction and is formed in a U shape, in which, the first side Z1 is opened when viewed in the axis O1 direction. For example, the attachment unit 71 is attached to the forceps insertion unit 21. In a case where the endoscope main body 16 of the sub-endoscope 10 is disposed in the attachment unit 71, the endoscope main body 16 is attached to the attachment unit 71.

In the forceps system 2 of the first modification example configured as described above, the attachment unit 71 attaches the endoscope main body 16 of the sub-endoscope 10 to the forceps 70, so that the operator such as a doctor can operate the forceps 70 and the endoscope main body 16 integrally.

In the forceps system of a second modification example, the entire opening 24a of the first small-diameter cylindrical portion 24 is disposed on any side (the first side Z1 or the second side Z2) perpendicular to the second reference plane S2, with respect to the second reference plane S2.

In the forceps system of the second modification example, the endoscope insertion unit 11 observes the distal end side with respect to the large-diameter cylindrical portion 22 through the opening 24a of the first small-diameter cylindrical portion 24. Then, in a case where the predetermined treatment is performed by opening and closing the gripping pieces 49A and 49B, the entire opening 24a is disposed on any side perpendicular to the second reference plane S2 with respect to the second reference plane S2 on which the distal end portions 49aA and 49aB of the gripping pieces 49A and 49B are opened and closed. Therefore, when the observation is performed by the endoscope insertion unit 11 through the opening 24a, it is possible to prevent the distal end portions 49aA and 49aB of the gripping pieces 49A and 49B from interfering with the observation.

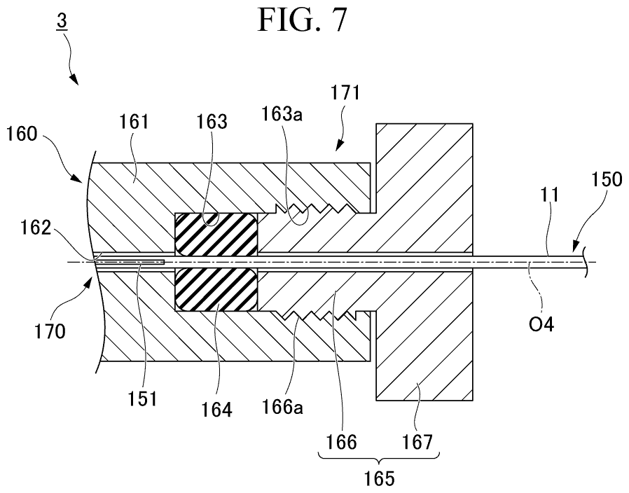

A forceps system 3 according to a third modification example shown in FIG. 7 includes a sub-endoscope 150, and a forceps 160 according to the present embodiment.

For example, a protruding strip 151 that protrudes outward from the endoscope insertion unit 11 is formed on an outer peripheral surface of the endoscope insertion unit 11 of the sub-endoscope 150. The protruding strip 151 extends along the endoscope insertion unit 11.

A small-diameter hole 162 and a large-diameter hole 163 are formed in a portion of a first small-diameter cylindrical portion 161 of the forceps 160, into which the endoscope insertion unit 11 is inserted. An inner diameter of the small-diameter hole 162 is smaller than an inner diameter of the large-diameter hole 163. The large-diameter hole 163 is disposed on the proximal end side with respect to the small-diameter hole 162.

A key groove (not shown) that fits into the protruding strip 151 of the endoscope insertion unit 11 is formed on an inner peripheral surface of the small-diameter hole 162. The key groove extends along the first axis O4 of the first small-diameter cylindrical portion 161. The protruding strip 151 and the key groove constitute a rotation regulation unit 170 that regulates the direction of the endoscope insertion unit 11 around the first axis O4.

A female screw 163a is formed on an inner peripheral surface of a proximal end side portion of the large-diameter hole 163. A tubular pressing member 164 is disposed in a distal end side portion in the large-diameter hole 163. For example, the pressing member 164 can be formed of synthetic rubber or the like.

The fixing member 165 is fitted with a female screw 163a of the first small-diameter cylindrical portion 161. The large-diameter hole 163, the pressing member 164, and the fixing member 165 of the first small-diameter cylindrical portion 161 constitute an insertion regulation unit 171.

The fixing member 165 includes a cylindrical portion 166 and a flange portion 167.

A male screw 166a that fits with the female screw 163a is formed on an outer peripheral surface of a distal end side portion of the cylindrical portion 166. The flange portion 167 protrudes from a proximal end side portion in the cylindrical portion 166 toward the outside.

For example, the fixing member 165 is formed of stainless steel or the like.

The endoscope insertion units 11 are each disposed in a cylindrical hole of the cylindrical portion 166 of the fixing member 165, a cylindrical hole of the pressing member 164, and the small-diameter hole 162 of the first small-diameter cylindrical portion 161.

In a case where the fixing member 165 that is fitted with the female screw 163a of the first small-diameter cylindrical portion 161 compresses the pressing member 164 in a first axis O4 direction, the inner diameter of the pressing member 164 is reduced, and the pressing member 164 presses the endoscope insertion unit 11 from the outside over the entire periphery. Therefore, the position of the endoscope insertion unit 11 disposed in the first small-diameter cylindrical portion 161 in the first axis O4 direction is regulated.

In the forceps system 3 of the third modification example configured as described above, the rotation regulation unit 170 can suppress change in a direction of an external field of view by the endoscope unit 11 due to the rotation of the endoscope insertion unit 11 around the first axis O4.

In addition, the insertion regulation unit 171 can suppress the change in the position of the endoscope insertion unit 11 in the first axis O4 direction with respect to the first small-diameter cylindrical portion 161.

It should be noted that the forceps system 3 may include one of the rotation regulation unit 170 and the insertion regulation unit 171.

While preferred embodiments of the invention have been described and illustrated above, it should be understood that these are exemplary of the invention and are not to be considered as limiting. Additions, omissions, substitutions, and other modifications can be made without departing from the scope of the invention.

Accordingly, the invention is not to be considered as being limited by the foregoing description and is only limited by the scope of the appended claims.

For example, in the embodiment and the modification example, the forceps 20, 70, and 160 may not include at least one of the opening and closing regulation unit 56 and the rotating operation unit 61. The forceps insertion unit 21 may not include at least one of the fixing unit 23, the second small-diameter cylindrical portion 25, or the bracket 26.

In a case where the forceps 20, 70, and 160 do not include the second small-diameter cylindrical portion 25, the two brackets 26 of the forceps insertion unit 21 may hold the position of the operation member 45 with respect to the large-diameter cylindrical portion 22.

### BRIEF DESCRIPTION OF THE REFERENCE SYMBOLS

1, 2, 3: forceps system
10, 150: sub-endoscope (endoscope)
11: endoscope insertion unit
20, 70, 160: forceps
21: forceps insertion unit
22: large-diameter cylindrical portion
23: fixing unit
24, 161: first small-diameter cylindrical portion (small-diameter cylindrical portion)
24a: opening
41: treatment unit
49A, 49B: gripping piece
56: opening and closing regulation unit
61: rotating operation unit
71: attachment unit
170: rotation regulation unit
171: insertion regulation unit
O1: axis
O4: first axis (small-diameter axis)
O5: second axis (treatment axis)
S2: second reference plane (reference plane)

The invention claimed is:

1. A forceps comprising:
a forceps insertion unit; and
a treatment unit configured to perform a predetermined treatment, the treatment unit being disposed in the forceps insertion unit and protruding toward a distal end side with respect to the forceps insertion unit,
wherein:
the forceps insertion unit includes:
a large-diameter cylindrical portion in which a proximal end side portion of the treatment unit is disposed, and
a small-diameter cylindrical portion which is disposed in the large-diameter cylindrical portion and into which an endoscope insertion unit for observing an outside of an endoscope is insertable,
the treatment unit includes two gripping pieces that are openable and closable relative to each other,
distal end portions of the two gripping pieces are opened and closed on a reference plane along an axis of the large-diameter cylindrical portion, and
an entire opening at a distal end of the small-diameter cylindrical portion is disposed on only one side perpendicular to the reference plane with respect to the reference plane.

2. The forceps according to claim 1, further comprising:
an opening and closing regulation unit configured to regulate an opening and closing range of the two gripping pieces.

3. The forceps according to claim 1, wherein:
the forceps insertion unit includes a fixing unit that is disposed at an intermediate portion of the large-diameter cylindrical portion in an axis direction of the large-diameter cylindrical portion and fixes a position of the small-diameter cylindrical portion with respect to the large-diameter cylindrical portion.

4. The forceps according to claim 1, wherein:
the treatment unit extends along a treatment axis along the large-diameter cylindrical portion, and
the forceps further comprises a rotating operation unit configured to rotate the treatment unit around the treatment axis.

5. The forceps according to claim 1, further comprising:
an attachment unit to which an endoscope main body of the endoscope is attached.

6. The forceps according to claim 1, further comprising:
a rotation regulation unit configured to regulate a direction of the endoscope insertion unit around a small-diameter axis of the small-diameter cylindrical portion.

7. The forceps according to claim 1, further comprising:
an insertion regulation unit configured to regulate a position of the endoscope insertion unit disposed in the small-diameter cylindrical portion in a small-diameter axis direction of the small-diameter cylindrical portion.

8. A forceps system comprising:
the forceps according to claim 1; and
the endoscope.
